(19) United States
(12) Patent Application Publication
(10) Pub. No.: US 2004/0176463 A1
(43) Pub. Date: Sep. 9, 2004

(54) Immediate release formulation of n-(2-propylpentanoyl)glycinamide

(76) Inventors: Daniella Licht, Ramat Ilan Givat Shmuel (IL); Suher Abd-Elhai, Tira (IL); Rachel Cohen, Hadera (IL); Mazzi Dagan-Lion, Modiin (IL); Adrian Gilbert, Rananna (IL); Noa Leibovitch, Ramat-Ha Sharon (IL); Sasson Cohen, Tel Aviv (IL); Ruth Levy, Tel Aviv (IL)

Correspondence Address: John P. White, Cooper & Dunham LLP, 1185 Avenue of the Americas, New York, NY, 10036, US

(21) Appl. No.: 10/772,911

(22) Filed: Feb. 5, 2004

Related U.S. Application Data
(60) Provisional application No. 60/445,327, filed on Feb. 5, 2003.

(51) Int. Cl. A61K 31/19; A61K 31/16; A61K 9/20

(52) U.S. Cl. 514/616; 424/465; 514/557

(57) ABSTRACT

The subject invention provides an immediate release tablet comprising the following components: a) a uniform admixture of an active ingredient selected from the group consisting of valproic sodium acid, a pharmaceutically acceptable salt or ester of valproic acid, divalproex sodium, valpromide and a compound having the structure:

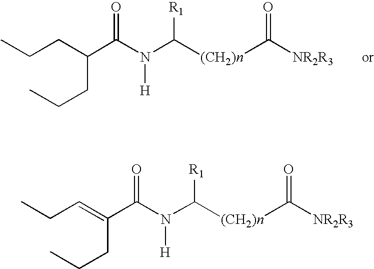

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and a hydroxypropyl cellulose, and b) a disintegrant, a process for manufacturing the tablet and a method of treating neuropathic pain, epilepsy, mania in bipolar disorder, a headache disorder, pain or of effecting pain prophylaxis in a subject.

This application claims the benefit of U.S. Provisional Application No. 60/445,327, filed Feb. 5, 2003, the entire contents of which are hereby incorporated by reference.

Throughout this application, various publications are referenced by full citations. The disclosures of these publications in their entireties are hereby incorporated by reference into this application in order to more fully describe the state of the art as known to those skilled therein as of the date of the invention described and claimed herein.

### BACKGROUND OF THE INVENTION

Pain is considered to play a basic physiological role in the detection and localization of tissue damage or potentially damaging physiological processes. Pain has been broadly classified as somatogenic, where a physiological explanation can be found, or psychogenic, where the physiological explanation is not known (The Merck Manual of Diagnosis and Therapy, 16^{th} Ed., pp. 1407-1426; PCT International Publication No. WO 02/13766 A2). An example of somatogenic pain is neuropathic pain.

Neuropathic pain is a category of pain, which includes several forms of non-nociceptive chronic pain, which result from dysfunction of nervous rather than somatic tissue. The majority of non-nociceptive chronic pains, in terms of either syndromes or cases, follow at various times after damage to either central or peripheral nervous tissue. Diagnosis of most of these syndromes and cases reveals a dependence on abnormal spatial and temporal summation of natural somatic stimulation in the spinal cord and independence from somatic disease and peripheral sympathetic nervous system activity. The scientific pain research community defines this kind of pain as centrally mediated neuropathic pain and recognizes mechanistic, diagnostic, and therapeutic commonalities among pains of this class and differences between these and other syndromes.

Neuropathic pain can be defined as pain deriving from damage to or inflammation of central or peripheral nervous system tissue. Examples of pain syndromes of this class include post herpetic neuralgia, neuritis, temporomandibular disorder, myofascial pain, back pain, and pain induced by inflammatory conditions. Neuropathic pain may occur in all body regions. For example, the pain may originate from the dental region.

Burn injury also often leads to neuropathic hyperalgesia in the affected body area. Neuralgia is characterized, in its acute phase, by intraneural inflammation, which can cause damage to primary afferent axons, thus inducing neuropathic pain. Neuropathic pain may also be induced by diabetic conditions (diabetic neuropathy). Neuropathy of primary afferent axons in long nerves is found in diabetic patients. Nociceptor sensitization may ensue (U.S. Pat. No. 6,054,461).

Pain can be both chronic and acute, and can also be evoked by noxious stimuli, also referred to as hyperalgesia, or by non-noxious stimuli referred to as allodynia (Attal, N. “Mechanism of action and rationale for use of antiepileptic drugs” (1999) in International Congress and Symposium Series 241 The Royal Society of Medicine Press, Limited Ed. J M Pellock). Allodynia and hyperalgesia can have mechanical causes (dynamic or static), or a thermal cause. Examples of neuropathic pain include all the painful peripheral neuropathies and specifically diabetic peripheral neuropathy, postherpetic neuralgia, and trigeminal neuralgia. Trigeminal neuralgia, for example, is the most common neuralgic syndrome in the elderly. Other types of somatogenic pain that may have neuropathic components include cancer pain, postoperative pain, lower back pain, complex regional pain syndrome, phantom pain, HIV pain, arthritis (osteo-arthritis and rheumatoid arthritis) pain and migraines.

Pain may also be a symptom of headache disorders. Migraines constitute one of the four major categories of primary headaches (International Headache Society, 1988; Silberstein, S. D. et al. Headache in Clinical Practice, (1998) Pub. Isis Medical Media, Oxford). The other three types of primary headaches are tension -type, cluster and a miscellaneous-type (Id.). One current view is that there is a continuous spectrum of headache severity ranging from mild tension headaches to severe migraines. Others consider tension headaches and migraines to be distinct entities.

Neuropathic pain conditions are characterized by hyperesthesia (enhanced sensitivity to a natural stimulus), hyperalgesia (abnormal sensitivity to pain), allodynia (widespread tenderness, characterized by hypersensitivity to tactile stimuli), and/or spontaneous burning pain. In humans, neuropathic pain tends to be chronic. Consequently, alternate therapies for the management of this form of chronic or neuropathic pain are widely sought. (U.S. Pat. No. 6,054,461).

The initial drug of choice for treating trigeminal neuralgia is carbamazepine. For other types of pain, such as postherpetic neuralgia and painful diabetic neuropathy, amitriptyline is most commonly used.

Drugs used in the treatment of headache disorders such as migraines originate from a broad range of different drug categories. These include: 5-hydroxytryptamine agonists (5-HT₁ agonists), dihydroergotamine, ergotamine, anti-emetics, anxiolytics, non-steroidal anti-inflammatory drugs, steroids, major tranquilizers, narcotics, beta-blockers, calcium channel blockers, anti-depressants, and anti-epileptic drugs. However, not all of the drugs in these categories are truly effective. While there are some drugs which are effective, there is still a need for more efficacious drugs, as well as a need for antimigraine treatments with fewer side effects.

As neuropathic pain tends to be chronic, drug treatment needs to be administered several times daily. The same is true for treating epilepsy. Epilepsy is an ancient disease, which affects about 1% of the global population. Despite the progress made in antiepileptic drug therapy, there are still many patients who continue to suffer from uncontrolled seizures and medication toxicity. At present, only the following 4 major antiepileptic drugs are in use: phenobarbital, phenytoin, carbamazepine and valproic acid. About 25% of the patient population is not seizure-free while treated with these medications (both mono and polytherapy), even when diagnosis and therapy is optimal (“Sustained Release Formulations of Antiepileptics” Clin. Pharmacokinet. (1992) 22(1): 11-24).

| Drug | Introduction (US Market) |
|---|---|
| Phenobarbital | 1912 |
| Phenytoin | 1938 |
| Carbamazepine | 1968 |
| Valporate | 1978 |

In addition, uncontrolled epilepsy is a significant problem, as approximately 20% of patients do not respond to traditional therapies.

Valproic acid (VPA) is an anticonvulsant in both its spectrum of activity (tonic, atonic and myoclonic seizures, atypical absence) and its chemical structure. However, its chemical structure is unrelated to the structural features common in other anticonvulsants.

The basis of valproic acid's anticonvulsant activity has not been unequivocally determined. However, it is believed to be related to its ability to block sodium channels and to increase GABA concentration in the brain by enhancing GABA release from GABA-ergic neurons and inhibiting its metabolism.

VPA therapy has been associated with several side effects, of which the most common are GI side effects, pancreatitis, weight gain, hepatoxicity and teratogenicity.

Bialer, et al. (U.S. Pat. No. 5,585,358) disclose derivatives of Valproic acid amides and 2-Valpronoic acid amides, methods of making and pharmaceutical compositions comprising these compounds. The compositions are disclosed in tablet, suppository and solution forms, but the details of the manufacturing process are not disclosed.

N-(2-Propylpentanoyl)glycinamide is an anti-epilepsy and anti-pain drug which has the structure:

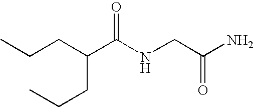

and can be prepared as disclosed by Bialer et al. in U.S. Pat. No. 5,585,358. U.S. Pat. No. 5,585,358 also describes a series of derivatives of valproic acid amides and 2-valproenic acid amides for the treatment of epilepsy and other neurological disorders.

Bialer et al. refer to the above compound as N-(2-n-Propylpentanoyl)glycinamide. However, in the present application, the compound is referred to as N-(2-Propylpentanoyl)glycinamide.

Published U.S. Patent Application No. US-2002-0052418-A1 discloses the use of N-(2-Propylpentanoyl)glycinamide and other derivatives of valproic acid amides and 2-valproenic acid amides for the treatment or prevention of pain and/or headache disorders.

The present invention provides an immediate release pharmaceutical composition comprising the active-material N-(2-propylpentanoyl)glycinamide and a method of manufacturing the composition wherein the composition contains a large dose of the active material.

### SUMMARY OF INVENTION

The subject invention provides an immediate release solid dosage form comprising the following components:

a) a uniform admixture of:

(i) an active ingredient selected from the group consisting of valproic sodium acid, a pharmaceutically acceptable salt or ester of valproic acid, divalproex sodium, valpromide and a compound having the structure:

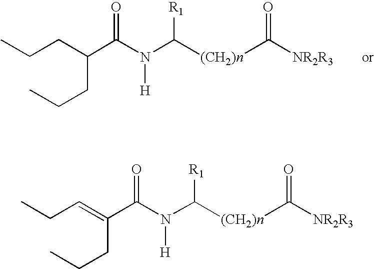

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a hydroxypropyl cellulose, and

b) a disintegrant.

The subject invention also provides an immediate release tablet comprising the following components:

a) a uniform admixture of:

(i) N-(2-Propylpentanoyl)glycinamide; and

(ii) a hydroxypropyl cellulose; and

b) a disintegrant.

### DETAILED DESCRIPTION OF THE FIGURES

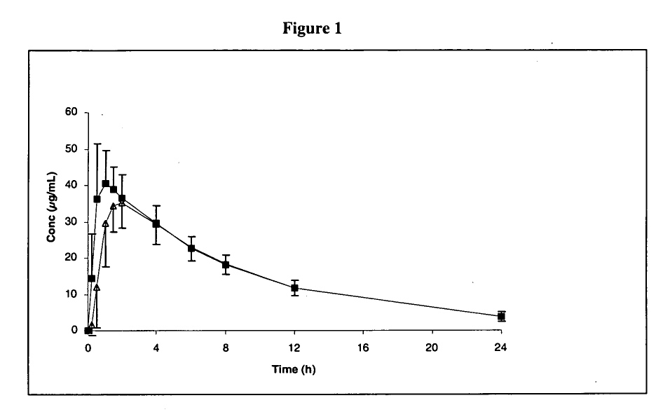

FIG. 1 shows Mean plasma (SD) concentration of N-(2-propylpentanoyl) glycinamide after single oral administration of 1500 mg (3×500 mg) N-(2-propylpentanoyl) glycinamide under fasting or fed conditions.

-▪- fasting state

-Δ- fed state

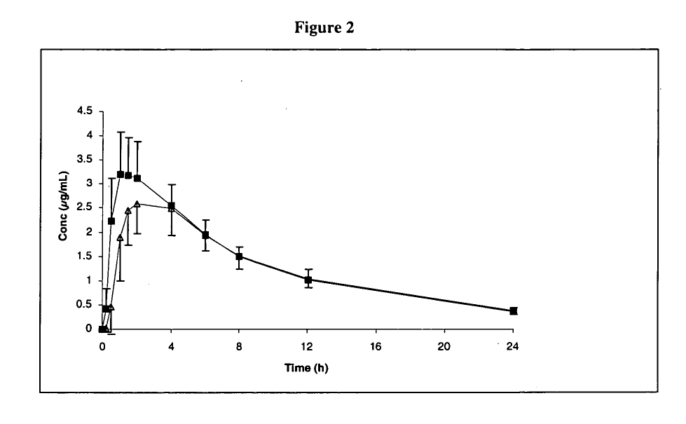

FIG. 2 shows mean plasma (SD) concentration of N-(2-propylpentanoyl) glycine after single oral administration of 1500 mg (3×500 mg) N-(2-propylpentanoyl) glycinamide under fasting or fed conditions.

-▪- fasting state

-Δ- fed state

### DETAILED DESCRIPTION

The subject invention provides an immediate release solid dosage form comprising the following components:

a) a uniform admixture of:

(i) an active ingredient selected from the group consisting of valproic sodium acid, a pharmaceutically acceptable salt or ester of valproic acid, divalproex sodium, valpromide and a compound having the structure:

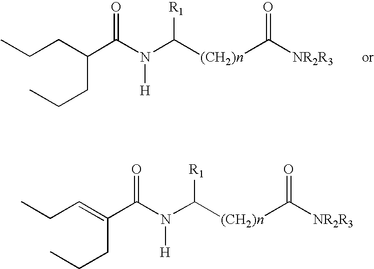

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a hydroxypropyl cellulose, and

b) a disintegrant.

In one embodiment, the solid dosage form is a tablet.

In one embodiment, the uniform admixture of component a) further comprises a filler.

In another embodiment, the solid dosage form further comprises a filler and a lubricant as additional components.

In a further embodiment, the filler of component a) is a microcrystalline cellulose, lactose, a starch, or a combination of two or more of the foregoing.

In a further embodiment, the filler of component a) is a microcrystalline cellulose.

In a further embodiment, the additional filler is a microcrystalline cellulose, lactose, a starch, or a combination of two or more of the foregoing.

In a further embodiment, the additional filler is a microcrystalline cellulose.

In a further embodiment, the additional filler is lactose.

In another embodiment, the lubricant is magnesium stearate, sodium stearyl fumarate, hydrogenated castor oil, hydrogenated soybean oil, polyethylene glycol or a combination of two or more of the foregoing.

In a further embodiment, the lubricant is magnesium stearate.

In another embodiment, the lubricant is sodium stearyl fumarate.

In another embodiment, the disintegrant of component b) is croscarmellose sodium, sodium starch glycolate or a combination thereof.

In a further embodiment, the disintegrant of component b) is croscarmellose sodium.

In another embodiment, the active ingredient of component a) is selected from the group consisting of valproic sodium acid, a pharmaceutically acceptable salt or ester of valproic acid, divalproex sodium, valpromide,

N-(2-Propylpentanoyl)glycinamide,

N-(2-propylpentanoyl)glycine-N′-methylamide,

N-(2-propylpentanoyl)glycine-N′-butylamide,

N-(2-propylpentanoyl)leucinamide,

N-(2-propylpentanoyl)alanine-N′-benzylamide,

N-(2-propylpentanoyl)alapinamide,

N-(2-propylpentanoyl)-2-phenylglycinamide,

N-(2-propylpentanoyl)threoninamide,

N-(2-propylpentanoyl)glycine-N′,N′-dimethylamide,

N-(2-propylpent-2-enoyl)glycinamide,

N-(2-propylpent-2-enoyl)alaninamide, and

N-(2-propylpent-2-enoyl)glycine-N′-methylamide.

The subject invention also provides an immediate release tablet comprising the following components:

a) a uniform admixture of:

(i) N-(2-Propylpentanoyl)glycinamide; and

(ii) a hydroxypropyl cellulose; and

b) a disintegrant.

In one embodiment, the uniform admixture of component a) further comprises a filler, and the tablet further comprises a filler and a lubricant as additional components.

In one embodiment, the filler of component a) is a microcrystalline cellulose, lactose, a starch, or a combination of two or more of the foregoing.

In a further embodiment, the filler of component a) is a microcrystalline cellulose.

In another embodiment, the additional filler is a microcrystalline cellulose, lactose, a starch, or a combination of two or more of the foregoing.

In a further embodiment, the additional filler is a microcrystalline cellulose.

In another embodiment, the additional filler is lactose.

In another embodiment, the lubricant is magnesium stearate, sodium stearyl fumarate, hydrogenated castor oil, hydrogenated soybean oil, polyethylene glycol or a combination of two or more of the foregoing.

In a further embodiment, the lubricant is magnesium stearate.

In a further embodiment, the lubricant is sodium stearyl fumarate.

In a further embodiment, the disintegrant of component b) is croscarmellose sodium, sodium starch glycolate or a combination thereof.

In another embodiment, the disintegrant of component b) is croscarmellose sodium.

In another embodiment, the tablet comprises the following components:

a) a uniform admixture of

from 50 mg/tablet to 1000 mg/tablet N-(2-Propylpentanoyl)glycinamide; and

from 5 mg/tablet to 150 mg/tablet hydroxypropyl cellulose; and

b) from 1 mg/tablet to 100 mg/tablet croscarmellose sodium.

In another embodiment, component a) further comprises from 1 mg/tablet to 300 mg/tablet microcrystalline cellulose as an additional component.

In another embodiment, component b) further comprises

from 5 mg/tablet to 500 mg/tablet filler; and

from 0.1 mg/tablet to 20 mg/tablet lubricant.

In another embodiment, the tablet comprises the following components:

a) a uniform admixture of

from 250 mg/tablet to 500 mg/tablet N-(2-Propylpentanoyl)glycinamide; and

from 25 mg/tablet to 50 mg/tablet hydroxypropyl cellulose; and

b) from 40 mg/tablet to 60 mg/tablet croscarmellose sodium.

In another embodiment, component a) further comprises from 50 mg/tablet to 100 mg/tablet microcrystalline cellulose as an additional component.

In another embodiment, component b) further comprises

from 100 mg/tablet to 500 mg/tablet filler; and

from 2 mg/tablet to 20 mg/tablet lubricant.

In a further embodiment, component b) comprises

from 5 mg/tablet to 20 mg/tablet lubricant.

In a further embodiment, component b) comprises

from 10 mg/tablet to 20 mg/tablet.

In a further embodiment, component b) comprises

from 15 mg/tablet to 20 mg/tablet.

In a further embodiment, component b) comprises

from 150 mg/tablet to 500 mg/tablet filler.

In a further embodiment, component b) comprises

from 200 mg/tablet to 500 mg/tablet filler.

In a further embodiment, component b) comprises

from 250 mg/tablet to 500 mg/tablet filler.

In a further embodiment, component b) comprises

from 300 mg/tablet to 500 mg/tablet filler.

In a further embodiment, component b) comprises

from 350 mg/tablet to 500 mg/tablet filler.

In a further embodiment, component b) comprises

from 400 mg/tablet to 500 mg/tablet filler.

In a further embodiment, component b) comprises

from 450 mg/tablet to 500 mg/tablet filler.

In a further embodiment, component b) comprises any combination of the aforementioned ranges of filler and lubricant.

In another embodiment,

the additional filler is lactose, microcrystalline cellulose, mannitol or a combination of two or more of the foregoing; and

the lubricant is magnesium stearate or sodium stearyl fumarate or a combination thereof.

In another embodiment, the tablet comprises the following components:

a) a uniform admixture of

500 mg/tablet N-(2-Propylpentanoyl) glycinamide;

50 mg/tablet hydroxypropyl cellulose; and

100 mg/tablet a microcrystalline cellulose, and

b) 55 mg/tablet croscarmellose sodium;

145 mg/tablet lactose; and

6 mg/tablet magnesium stearate.

In another embodiment, the tablet comprises the following components:

a) a uniform admixture of

500 mg/tablet N-(2-Propylpentanoyl) glycinamide;

50 mg/tablet hydroxypropyl cellulose; and

100 mg/tablet a microcrystalline cellulose, and

b) 50 mg/tablet croscarmellose sodium;

145 mg/tablet lactose; and

6 mg/tablet magnesium stearate.

In another embodiment, the tablet comprises

a) a uniform admixture of:

250 mg/tablet N-(2-Propylpentanoyl) glycinamide;

25 mg/tablet hydroxypropyl cellulose; and

50 mg/tablet microcrystalline cellulose;

b) 450 mg/tablet microcrystalline cellulose;

50 mg/tablet croscarmellose sodium; and

6 mg/tablet magnesium stearate.

The subject invention also provides a method of treating neuropathic pain in a subject in need of such treatment comprising administering to the subject a therapeutically effective dose of any of the solid dosage forms or tablets of the invention in order to thereby treat the neuropathic pain in the subject.

The subject invention also provides a method of treating a headache disorder in a subject in need of such treatment comprising administering to the subject a therapeutically effective dose of any of the solid dosage forms or tablets of the invention in order to thereby treat the headache disorder in the subject.

The subject invention also provides a method of treating epilepsy in a subject in need of such treatment comprising administering to the subject a therapeutically effective dose of any of the solid dosage forms or tablets of the invention in order to thereby treat epilepsy in the subject.

The subject invention also provides a method of controlling seizures in a subject suffering from epilepsy comprising administering to the subject a therapeutically effective dose of any of the solid dosage forms or tablets of the invention in order to thereby control the seizures in the subject.

The subject invention also provides a method of treating pain in a subject in need of such treatment comprising administering to the subject a therapeutically effective dose of any of the tablets of the invention in order to thereby treat pain in the subject.

The subject invention also provides a method of pain prophylaxis in a subject in need of such treatment comprising administering to the subject a prophylactic dose of any of the tablets of the invention in order to thereby effect pain prophylaxis in the subject.

The subject invention also provides a method of treating mania in bipolar disorder in a subject in need of such treatment comprising administering to the subject a therapeutically effective dose of any of the tablets of the invention in order to thereby treat mania in bipolar disorder in the subject.

The subject invention also provides a method of attenuating bipolar mood swings in a subject suffering from bipolar disorder comprising administering to the subject a therapeutically effective dose of any of the solid dosage forms or tablets of the invention in order to thereby attenuate the bipolar mood swings in the subject.

The subject invention also provides a process for preparing the solid dosage form or tablet of the invention, comprising the steps of:

a) admixing predetermined amounts of

(i) an active ingredient selected from the group consisting of valproic sodium acid, a pharmaceutically acceptable salt or ester of valproic acid, divalproex sodium, valpromide and a compound having the structure:

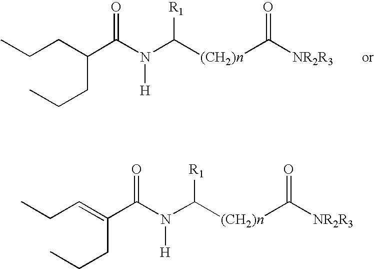

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a hydroxypropyl cellulose;

b) admixing the uniform mixture of step a) with a predetermined amount of a disintegrant; and

c) compressing the mixture of step b) to form the tablet.

In one embodiment, step b) further comprises admixing the uniform mixture with predetermined amounts of a filler and a lubricant.

In another embodiment, the filler of step b) is microcrystalline cellulose, anhydrous dicalcium phosphate, lactose or a combination of two or more of the foregoing.

In a further embodiment, the filler is lactose.

In a further embodiment, the filler is a microcrystalline cellulose.

In another embodiment, the lubricant is magnesium stearate or sodium stearyl fumarate or a combination thereof.

In a further embodiment, the lubricant is magnesium stearate.

In a further embodiment, the lubricant is sodium stearyl fumarate.

In another embodiment, the disintegrant of step b) is croscarmellose sodium, sodium starch glycolate or a combination thereof.

In a further embodiment, the disintegrant of step b) is croscarmellose sodium.

The subject invention also provides the use of an active ingredient selected from the group consisting of valproic sodium acid, a pharmaceutically acceptable salt or ester of valproic acid, divalproex sodium, valpromide and a compound having the structure:

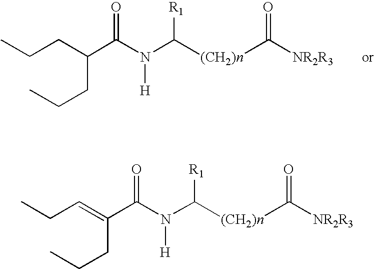

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in treating a headache disorder in a subject.

The subject invention also provides the use of an active ingredient selected from the group consisting of valproic sodium acid, a pharmaceutically acceptable salt or ester of valproic acid, divalproex sodium, valpromide and a compound having the structure:

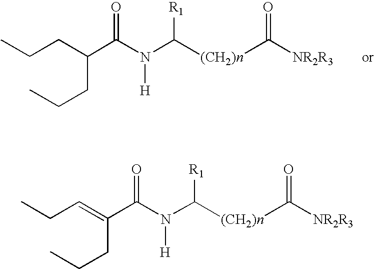

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in treating neuropathic pain in a subject.

The subject invention also provides the use of an active ingredient selected from the group consisting of valproic sodium acid, a pharmaceutically acceptable salt or ester of valproic acid, divalproex sodium, valpromide and a compound having the structure:

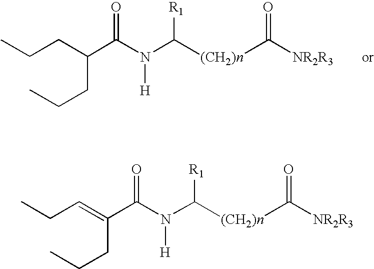

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in treating epilepsy in a subject.

The subject invention also provides the use of an active ingredient selected from the group consisting of valproic sodium acid, a pharmaceutically acceptable salt or ester of valproic acid, divalproex sodium, valpromide and a compound having the structure:

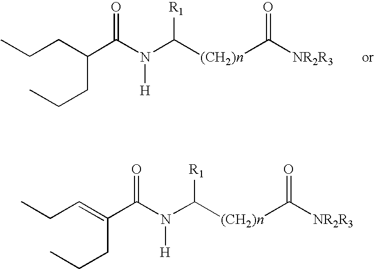

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in controlling seizures in a subject suffering from epilepsy.

The subject invention also provides the use of an active ingredient selected from the group consisting of valproic sodium acid, a pharmaceutically acceptable salt or ester of valproic acid, divalproex sodium, valpromide and a compound having the structure:

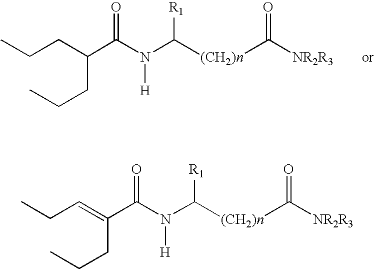

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in treating mania in bipolar disorder in a subject.

The subject invention also provides the use of an active ingredient selected from the group consisting of valproic sodium acid, a pharmaceutically acceptable salt or ester of valproic acid, divalproex sodium, valpromide and a compound having the structure:

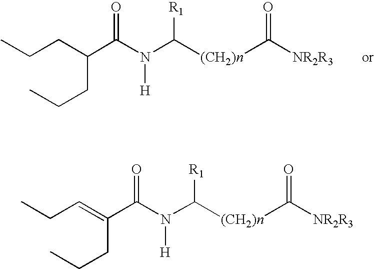

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in attenuating bipolar mood swings in a subject suffering from bipolar disorder.

The subject invention also provides the use of an active ingredient selected from the group consisting of valproic sodium acid, a pharmaceutically acceptable salt or ester of valproic acid, divalproex sodium, valpromide and a compound having the structure:

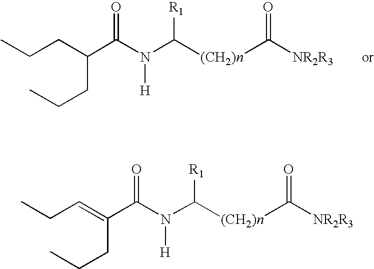

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in treating pain in a subject.

The subject invention also provides the use of an active ingredient selected from the group consisting of valproic sodium acid, a pharmaceutically acceptable salt or ester of valproic acid, divalproex sodium, valpromide and a compound having the structure:

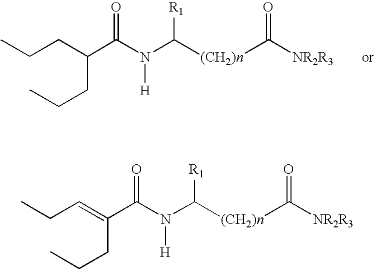

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing any of the immediate release solid dosage forms or tablets of the invention for use in effecting pain prophylaxis in a subject.

The subject invention also provides any of the above immediate release solid dosage forms or tablets for use in treating a headache disorder in a subject.

The subject invention also provides any of the above immediate release solid dosage forms or tablets for use in treating neuropathic pain in a subject.

The subject invention also provides any of the above immediate release solid dosage forms or tablets for use in treating epilepsy in a subject.

The subject invention also provides any of the above immediate release solid dosage forms or tablets for use in controlling seizures in a subject suffering from epilepsy.

The subject invention also provides any of the above immediate release solid dosage forms or tablets for use in treating mania in bipolar disorder in a subject.

The subject invention also provides any of the above immediate release solid dosage forms or tablets for use in attenuating bipolar mood swings in a subject suffering from bipolar disorder.

The subject invention also provides any of the above immediate release solid dosage forms or tablets for use in treating pain in a subject.

The subject invention also provides any of the above immediate release solid dosage forms or tablets for use in effecting pain prophylaxis in a subject.

The subject invention provides an oral dosage of N-(2-propylpentanoyl)glycinamide in an immediate release form.

In one embodiment of the invention, the process for manufacturing the immediate release formulation of N-(2-propylpentanoyl)glycinamide comprises:

1. Preparing a granulate of N-(2-propylpentanoyl) glycinamide;

2. Mixing the granulate of step 1 with excipients; and

3. Compressing the mixture of step 2 to form an immediate release tablet of N-(2-propylpentanoyl) glycinamide.

In another embodiment, the process for manufacturing the immediate release formulation of N-(2-propylpentanoyl) glycinamide comprises:

1. Mixing the active material with excipients; and

2. Direct compression of the mixture of step 1.

As used herein, the phrase, “immediate release” indicates that the drug is allowed to dissolve in the gastrointestinal tract, with no intention of delaying or prolonging the dissolution or absorption of the drug (FDA Guideline for industry SUPAC-MR: modified release oral dosage forms CDER, September, 1997). Immediate release formulations encompass, for example, rapid burst formulations.

Non-limiting examples of disintegrants used in the subject invention are kaolin starch, powdered sugar, sodium starch glycolate, crosscaramelose sodium, microcrystalline cellulose, carboxymethyl cellulose and sodium alginate.

Non-limiting examples of a filler used in the subject invention (used for example for weight adjustment and for better compression) are corn starch, lactose, glucose, various natural gums, methylcellulose, carboxymethylcellulose, microcrystalline cellulose, calcium phosphate, calcium carbonate, calcium sulfate kaolin, sodium chloride, powdered cellulose, sucrose, mannitol and starch.

Non-limiting examples of a binding agent used in the subject invention (used for example for the granulate) are alginic acid, acia, carbomer, carboxymethylcellulose sodium, dextrin, ethylcellulose, gelatin, guar gum, hydrogenated vegetable oil, hydroxyethylcellulose, hydroxypropylcellulose (e.g.; Klucel®, Aqualon Division, Hercules Incorporated, Wilmington, Del.), hydroxypropylmethylcellulose, liquid glucose, magnesium aluminum silicate, maldodextrin, methylcellulose, polymethacrylates, povidone (polyvinylpyrrolidone), pregelatinized starch, sodium alginate, starch, and zein. In a preferred embodiment, the excipient used as a binding agent comprises a hydroxypropylcellulose.

In one embodiment, the excipient used as a binder is hydroxypropyl cellulose. In one embodiment, the hydroxypropyl cellulose has a particle size distribution such that about 85% of the hydroxypropyl cellulose passes through a 30 mesh screen. In another embodiment, the hydroxypropyl cellulose has a particle size distribution such that about 99% of the hydroxypropyl cellulose passes through a 20 mesh screen. In another embodiment, the hydroxypropyl cellulose has a pH of 5.0-7.5 in water solution. In one embodiment, the hydroxypropyl cellulose has an average molecular weight of 1,150,000. In one embodiment, the hydroxypropyl cellulose has an average molecular weight of 850,000. In one embodiment, the hydroxypropyl cellulose has an average molecular weight of 370,000. In one embodiment, the hydroxypropyl cellulose has an average molecular weight of 140,000. In one embodiment, the hydroxypropyl cellulose has an average molecular weight of 95,000. In one embodiment, the hydroxypropyl cellulose has an average molecular weight of 80,000. In one embodiment, the hydroxypropyl cellulose has a viscosity of 1,500-3,000 cps at a concentration of 1% by weight in water at 25° C. In one embodiment, the hydroxypropyl cellulose has a viscosity of 4,000-6,500 cps at a concentration of 2% by weight in water at 25° C. In one embodiment, the hydroxypropyl cellulose has a viscosity of 150-400 cps at a concentration of 2% by weight in water at 25° C. In one embodiment, the hydroxypropyl cellulose has a viscosity of 150-400 cps at a concentration of 5% by weight in water at 25° C. In one embodiment, the hydroxypropyl cellulose has a viscosity of 75-150 cps at a concentration of 5% by weight in water at 25° C. In one embodiment, the hydroxypropyl cellulose has a viscosity of 200-600 cps at a concentration of 10% by weight in water at 25° C. In one embodiment, the hydroxypropyl cellulose has a viscosity of 75-150 cps at a concentration of 5% by weight in water at 25° C. In one embodiment, the hydroxypropyl cellulose has a viscosity of 300-600 cps at a concentration of 10% by weight in water at 25° C.

In one embodiment, the excipient used as a filler is a microcrystalline cellulose. In an added embodiment, the microcrystalline cellulose has an average particle size between about 20 and about 200 microns. In an added embodiment, the microcrystalline cellulose has an average particle size between about 50 and about 90 microns.

Details of general formulation procedures and information on additional excipients may be found in Remington: The Science and Practice of Pharmacy, 20^{th} Edition.

This invention will be better understood from the Experimental Details which follow.

Experimental Details:

Two clinical trials, single and multiple dose, were performed in healthy male volunteers by administration of the N-(2-propylpentanoyl)glycinamide drug product in capsules.

In a subsequent clinical trial, the drug product was administered in caplet shaped tablets. In order to confirm that the capsule and tablet forms of the drug were equivalent, the properties of the two dosage forms were studied. The results of the study are presented below.

### EXAMPLE 1

### Comparison of Capsule and Caplet Formulations

The drug product used in this study was manufactured in two strengths. Each caplet contained 250 mg or 500 mg of N-(2-propylpentanoyl)glycinamide. Each capsule contained 250 mg or 50 mg of N-(2-propylpentanoyl)glycinamide.

TABLE 1
Composition of N-(2-propylpentanoyl)glycinamide
capsule (250 mg and 50 mg)

| | | |
|---|---|---|
| | N-(2- | N-(2- |
| | propylpentanoyl) | propylpentanoyl) |
| | glycinamide | glycinamide |
| Ingredients | Capsule 250 mg | Capsule 50 mg |

| | | | | |
|---|---|---|---|---|
| N-(2- | 250.0 | mg | 50.0 | mg |
| propylpentanoyl) | | | | |
| glycinamide | | | | |
| Lactose Spray | 67.5 | mg | 399.5 | mg |
| Dried | | | | |
| Aerosil 200* | 2.5 | mg | 0.5 | mg |

*Colloidal silicon dioxide NF

TABLE 2
Composition of N-(2-propylpentanoyl)glycinamide
caplets (250 mg)

| | mg per | | Reference to |
|---|---|---|---|
| Ingredients | caplet | Function | standards |
| N-(2- | 250 | Active | Teva Ref. |
| propylpentanoyl) | | Ingredient | standard |
| glycinamide | | | |
| Povidone USP | 30 | Binder | USB/BP |
| (PVP K-30) | | | |
| Sodium Starch | 25 | Disintegrate | NF/BP |
| Glycolate NF | | | |
| Lactose | 123.5 | Bulking | NF/BP |
| Monohydrate | | agent | |
| (200 Mesh) | | | |
| Starch NF | 50 | Binder | NF/BP |
| Glycerin USP | 2.5 | Humectant | USP |
| Avicel PH 102 | 246.5 | Diluent | NF/Ph. Eur/Jp |
| (Microcorystalline | | | |
| Cellulose | | | |
| Croscarmellose | 25 | Disintegrate | NF |
| Sodium | | | |
| (Ac-Di-Sol) | | | |
| Magnesium Stearate | 2.5 | Lubricant | NF/BP |
| Purified Water | | Moistener | USP |
| | | Removed | |
| | | during | |
| | | manufacture | |

TABLE 3
Composition of N-(2-propylpentanoyl)glycinamide
caplets (500 mg)

| | mg per | | Reference to |
|---|---|---|---|
| Ingredients | caplet | Function | standards |
| N-(2- | 500 | Active | Teva Ref. |
| propylpentanoyl) | | Ingredient | standard |
| glycinamide | | | |
| Povidone USP | 30 | Binder | USB/BP |
| (PVP K-30) | | | |
| Sodium Starch | 25 | Disintegrant | NF/BP |
| Glycolate NF | | | |
| Pregelatinized | 50 | Binder | NF |
| Starch (starch- | | | |
| STA-RX 1500 NF) | | | |
| Starch NF | 50 | Binder | NF/BP |
| Glycerin USP | 2.5 | Humectant | USP |
| Avicel PH 102 | 70 | Diluent | NF/Ph. Eur/Jp |
| (Microcrystalline | | | |
| Cellulose) | | | |
| Croscarmellose | 25 | Disintegrant | NF |
| Sodium (Ac-Di- | | | |
| Sol) | | | |
| Magnesium | 2.5 | Lubricant | NF/BP |
| Stearate | | | |
| Purified Water | | Moistener | USP |
| | | Removed | |
| | | during | |
| | | manufacture | |

Dissolution experiments using N-(2-propylpentanoyl) glycinamide capsules and N-(2-propylpentanoyl)glycinamide caplets (250 mg and 500 mg) exhibited a fast rate of dissolution. The percent dissolution after 10 minutes and after 45 minutes were as follows:

TABLE 3a
Percent dissolution

| | mg active per | | Dissolution after 10 | Dissolution after 45 |
|---|---|---|---|---|
| Dosage form | dosage form | | minutes | minutes |
| Capsules | 50 | mg | Not available | 96.9% |
| | 250 | mg | 86.6% | 99.0% |
| Caplets | 250 | mg | 78.8% | 94.8% |
| | 500 | mg | 101.4% | 93.7% |

The two different dosage forms of N-(2-propylpentanoyl)glycinamide studied above (capsules and caplets) were found to be equivalent based on dissolution data of both formulations presented above.

### EXAMPLE 2

### N-(2-propylpentanoyl)glycinamide granulate

In a further study, N-(2-propylpentanoyl)glycinamide was granulated with a binder solution and with several excipients. The granulate was then compressed into a tablet and the tablets were evaluated for their dissolution rates.

TABLE 4
Composition of the granulate

Mg/tablet

| Excipient | Use | A | B |
|---|---|---|---|
| N-(2-propylpentanoyl) | Active material | 500 | 250 |
| glycinamide | | | |
| Microcrystalline Cellulose | Filler | 100 | 50 |
| Hydroxypropyl cellulose | Binder | 50 | 25 |
| Total | | 650 | 325 |

TABLE 5
Composition of the tablets

Mg/tablet

| Excipient | Use | A | B |
|---|---|---|---|
| N-(2-propylpentanoyl) | | 650 | 325 |
| glycinamidegranulate | | | |
| Microcrystalline | | — | 450 |
| cellulose | | | |
| Lactose | Filler | 145 | — |
| Croscarmellose | Disintegrant | 50 | 50 |
| sodium | | | |
| Magnesium | Lubricant | 6.0 | 6.0 |
| Stearate | | | |

The tablets were prepared by mixing the granulate with several excipients (table 5). Each formulation was tested in a dissolution test using 900 ml purified water, 37° C., in App. 2 US Pharmacopoeia (USP), at 75 RPM.

TABLE 6
Dissolution of N-(2-propylpentanoyl)glycinamide tablets

Formula

| | | |
|---|---|---|
| Time(min.) | A | B |

% Dissolution

| | | |
|---|---|---|
| 10 | 97 | 94 |
| 15 | 98 | 96 |
| 30 | 98 | 96 |
| 45 | 98 | 96 |

As can be seen the two different dosages (A and B) of N-(2-propylpentanoyl)glycinamide gave the same dissolution profile. The two dosages also exhibited good compression properties.

### EXAMPLE 3

### Effect of Variation in the Composition of the Tablet

TABLE 7
Composition of the tablets

| | FORMULA | |
|---|---|---|
| USE | C | D |

Mg/tablet

PART I

| | | | |
|---|---|---|---|
| N-(2-propylpentanoyl) | | 250 | 500 |
| glycinamide | | | |
| Sodium Starch Glycolate NF | Disintegrant | 30 | 30 |
| Starch STA-RX 1500 | Disintegrant | 120 | 65 |
| Starch NF | Filler/Binder | 40 | 20 |
| Avicel PH 101 | Filler | 155 | — |
| (Microcrystalline cellulose) | | | |

PART II

| | | | |
|---|---|---|---|
| Klucel LF | Binder | 40 | 30 |

PART III

| | | | |
|---|---|---|---|
| Avicel PH 102 | Filler | 100 | 90 |
| (Microcrystalline cellulose) | | | |
| AC-DI-SOL(Croscarmellose sodium) | Disintegrant | 30 | 30 |
| Magnesium Stearate | Lubricant | 3 | 3 |

Formulations C and D, each containing different excipients than formulations A and B were tested in order to determine the effect of varying the composition of the granulate and tablet matrix on the dissolution rate and on the physical properties of the manufactured tablets.

Each formulation was tested in a dissolution test using 900 ml purified water 37° C., in App.2 US Pharmacopoeia (USP).

TABLE 8
Dissolution of tablets

Formula

| | | |
|---|---|---|
| Time(min.) | C | D |

% Dissolution

| | | |
|---|---|---|
| 10 | 98 | 94 |
| 15 | 101 | 94 |
| 30 | 101 | 95 |
| 45 | 101 | 95 |

As shown above, the dissolution profile was found to be dependent upon the specific formulations. However, the physical compression properties of formulations A and B were found to be much better than formulations C and D.

### EXAMPLE 4

### Effect of the Amount Loss on Drying (L.O.D.) in the Granulate

TABLE 9

| | | E | F | G | GG |
|---|---|---|---|---|---|
| Formula | USE | Mg/tablet | | | |
| N-(2- | | 650 | 650 | 650 | 650 |
| propylpentanoyl) | | | | | |
| glycinamide | | | | | |
| granulate | | | | | |
| Avicel PH 102 | Filler | 125 | 125 | 125 | 125 |
| (Microcrystalline | | | | | |
| cellulose) | | | | | |
| AC-DI-SOL | Disintegrant | 50 | 50 | 50 | 50 |
| (Croscarmellose | | | | | |
| sodium) | | | | | |
| Magnesium | Lubricant | 6.0 | 6.0 | 6.0 | 6.0 |
| Stearate | | | | | |
| L.O.D. | | 1.0 | 1.6 | 2.0 | 3.0 |

TABLE 10
Dissolution of formulations

Formula

| | | | |
|---|---|---|---|
| E | F | G | GG |

| | |
|---|---|
| Time(min.) | % Dissolution |

| | | | | |
|---|---|---|---|---|
| 10 | 84 | 90 | 80 | 91 |
| 15 | 101 | 98 | 91 | 104 |
| 30 | 103 | 100 | 99 | 108 |
| 45 | 103 | 100 | 102 | 108 |

As can be seen, the dissolution rate did not change due to change in the amounts of the L.O.D. The physical properties also remained the same and the tablets were compressible in this range of the L.O.D.

### EXAMPLE 5

### Effect of the Binder in the Granulate

TABLE 11

| Formula | Use | H | I |
|---|---|---|---|
| N-(2-propylpentanoyl) | | 500 | 500 |
| glycinamide | | | |
| Sodium Starch | Disintegrant | 25 | 30 |
| Glycolate NF | | | |
| Starch STA-RX 1500 | Disintegrant | 50 | 65 |
| Binder Excipients | | | |
| Starch NF | Filler/Binder | 25 | 20 |
| Klucel LF | Binder | — | 30 |
| Starch NF | Filler/Binder | 25 | — |
| Glycerin USP | | 2.5 | — |
| PVP-K-30 | Binder | 30 | — |
| (Povidone USP) | | | |

TABLE 12
Dissolution of formulation

Formula

| | |
|---|---|
| H | I |

| | |
|---|---|
| Time(min.) | % Dissolution |

| | | |
|---|---|---|
| 10 | 96 | 100 |
| 15 | 96 | 103 |
| 30 | 97 | 103 |
| 45 | 97 | 103 |

As shown above, changing the type of binder did not change the dissolution rate. However, it had an effect on the granulate's physical properties. In particular, compression was more readily accomplished when Klucel was used as a binder.

### EXAMPLE 6

### Effect of Lubricant Type on Dissolution Rate

TABLE 13a

| Formula | USE | J | K |
|---|---|---|---|
| N-(2-propylpentanoyl) | | 650 | 650 |
| glycinamide granulate | | | |
| LACTOSE | Filler | 145 | 145 |
| AC-DI-SOL | Disintegrant | 50 | 50 |
| (croscarmellose | | | |
| sodium) | | | |
| Magnesium Stearate | Lubricant | — | 6.0 |
| Pruv | Lubricant | 6.0 | — |

TABLE 13b

Formula

| | |
|---|---|
| J | K |

| | |
|---|---|
| Time (min) | % dissolution |

| | | |
|---|---|---|
| 10 | 86 | 97 |
| 15 | 98 | 98 |
| 30 | 97 | 98 |
| 45 | 97 | 98 |

Changing the type of the lubricant did not change the dissolution rate. However, the type of lubricant did have an effect on the physical properties of the tablets. Compression was more easily accomplished when Pruv was used as the lubricant.

### EXAMPLE 7

### Effect of Lubricant Amount on Dissolution Rate

TABLE 14
Lower dosage tablets (325 mg of granulate/tablet)

| Formula | USE | L | M | N |
|---|---|---|---|---|
| N-(2-propylpentanoyl) | | 325 | 325 | 325 |
| glycinamide granulate | | | | |
| avicel PH 102 | Filler | 450 | 450 | 450 |
| (microcrystalline | | | | |
| cellulose) | | | | |
| AC-DI-SOL | Disintegrant | 50 | 50 | 50 |
| (croscarmellose | | | | |
| sodium) | | | | |
| Magnesium Stearate | Lubricant | 4.5 | 6.0 | 7.5 |

Changing the amount of the lubricant did not change the dissolution rate of the lower dosage tablets.

TABLE 15
Higher dosage tablets (650 mg of granulate/tablet)

| Formula | USE | LL | MM | NN |
|---|---|---|---|---|
| N-(2-propylpentanoyl) | | 650 | 650 | 650 |
| glycinamide granulate | | | | |
| Lactose | Filler | 145 | 145 | 145 |
| AC-DI-SOL | Disintegrant | 50 | 50 | 50 |
| (croscarmellose | | | | |
| sodium) | | | | |
| Magnesium Stearate | Lubricant | 3 | 6 | 9 |

TABLE 16
Dissolution of formulation

Formula

| | | |
|---|---|---|
| LL | MM | NN |

| | |
|---|---|
| Time(min) | % Dissolution |

| | | | |
|---|---|---|---|
| 10 | 84 | 97 | 91 |
| 15 | 97 | 98 | 99 |
| 30 | 100 | 98 | 100 |
| 45 | 101 | 98 | 100 |

As shown above, changing the amount of the lubricant did not change the dissolution rate of the higher dosage tablets.

### EXAMPLE 8

### Effect of the Filler Material on the Tabletting Process

TABLE 17

| Formula | USE | O | P | Q |
|---|---|---|---|---|
| N-(2- | | 650 | 650 | 650 |
| propylpentanoyl) | | | | |
| glycinamide | | | | |
| granulate | | | | |
| avicel PH 102 | Filler | 125 | — | — |
| (microcrystalline | | | | |
| cellulose) | | | | |
| Mannitol | Filler | — | 125 | — |
| Lactose | Filler | — | — | 145 |
| AC-DI-SOL | Disintegrant | 50 | 50 | 50 |
| (croscarmellose | | | | |
| sodium) | | | | |
| Magnesium | Lubricant | 6.0 | 6.0 | 6.0 |
| Stearate | | | | |

TABLE 18
Dissolution of formulations

Formula

| | |
|---|---|
| O | Q |

| | |
|---|---|
| Time(min.) | % Dissolution |

| | | |
|---|---|---|
| 10 | 80 | 97 |
| 15 | 91 | 98 |
| 30 | 99 | 98 |
| 45 | 102 | 98 |

As shown above, changing the amount of the filler did not change the dissolution rate. However, changing the amount of filter had an effect on the physical properties of the compression. In particular, formulations which used lactose S.D. as the filler were more easily compressed.

### EXAMPLE 9

### Effect of the Amount of Disintegrate on the Dissolution Rate

TABLE 19

| Formula | USE | R | S | RR | SS |
|---|---|---|---|---|---|
| N-(2- | | 325 | 325 | 650 | 650 |
| propylpentanoyl) | | | | | |
| glycinamide | | | | | |
| granulate | | | | | |
| Lactose | Filler | — | — | 145 | 145 |
| Avicel PH 102 | Filler | 450 | 450 | — | — |
| (Microcrystalline | | | | | |
| cellulose) | | | | | |
| AC-DI-SOL | Disintegrant | 50 | 40 | 50 | 45 |
| (Croscarmellose | | | | | |
| sodium) | | | | | |
| Magnesium | Lubricant | 6.0 | 6.0 | 6.0 | 6.0 |
| Stearate | | | | | |

TABLE 20
Dissolution of formulation

Formula

| | |
|---|---|
| RR | SS |

| | |
|---|---|
| Time(min.) | % Dissolution |

| | | |
|---|---|---|
| 10 | 97 | 66 |
| 15 | 98 | 91 |
| 30 | 98 | 100 |
| 45 | 98 | 100 |

As shown above, the amount of disintegrant significantly effects the dissolution rate of the formulation for the first 10 minutes. However, After 15 minutes, this effect is no longer discernible.

### EXAMPLE 10

### Effect of the Milling of the Granulate on Dissolution Rate

TABLE 21

| | | V | W | X |
|---|---|---|---|---|
| | | (Screen of | (Screen of | (Screen of |
| Formula | USE | 0.8 mm) | 1.0 mm) | 1.2 mm) |
| N-(2- | | 650 | 650 | 650 |
| propylpentanoyl) | | | | |
| glycinamide | | | | |
| granulate | | | | |
| Lactose | Filler | 145 | 145 | 145 |
| AC-DI-SOL | Disintegrant | 50 | 50 | 50 |
| (croscarmellose | | | | |
| sodium) | | | | |
| Magnesium | Lubricant | 6.0 | 6.0 | 6.0 |
| Stearate | | | | |

TABLE 22
Dissolution of formulation

| | Formula | | |
|---|---|---|---|
| | V | W | X |
| Time(min.) | % Dissolution | | |
| 10 | 92 | 100 | 93 |
| 15 | 103 | 105 | 102 |
| 30 | 104 | 106 | 105 |
| 45 | 105 | 106 | 105 |

As shown above, three granulates milled to different sizes gave similar dissolution rates.

### EXAMPLE 11

### Effect of Amount of Filler on Dissolution Rate

TABLE 23

| | | |
|---|---|---|
| Y | Z | ZZ |

| | | |
|---|---|---|
| Formula | USE | Mg/tablet |

| | | | | |
|---|---|---|---|---|
| N-(2- | | 650 | 650 | 650 |
| propylpentanoyl) | | | | |
| glycinamide | | | | |
| granulate | | | | |
| Lactose | Filler | 145 | 140 | 150 |
| AC-DI-SOL | Disintegrant | 50 | 50 | 50 |
| (croscarmellose | | | | |
| sodium) | | | | |
| Magnesium | Lubricant | 6.0 | 6.0 | 6.0 |
| Stearate | | | | |

TABLE 24
Dissolution of formulation

| | Formula | | |
|---|---|---|---|
| | Y | Z | ZZ |
| Time(min.) | % Dissolution | | |
| 10 | 85 | 77 | 84 |
| 15 | 96 | 97 | 98 |
| 30 | 100 | 101 | 100 |
| 45 | 100 | 102 | 100 |

As illustrated above, the amount of filler had a negligible effect on the dissolution rate of the manufactured tablets.

### EXAMPLE 12

### Plasma Concentration of N-(2-propylpentanoyl)Glycinamide and of N-(2-propylpentanoyl)Glycine After Administration

Formulation A was prepared as described in Example 2.

Three tablets of formulation A (3×500 mg active pharmaceutical ingredient) were simultaneously administered to each of 32 healthy male and female volunteers. Plasma concentrations of N-(2-propylpentanoyl)glycinamide and of a major metabolite, N-(2-propylpentanoyl)glycine of each of the volunteers were regularly analyzed at 0.25, 0.5, 0.75, 1.0, 1.5, 2, 4, 6, 8, 12, and 24 hours. The tablets were administered once while the volunteers were under fed conditions and once while the volunteers were fasting. Between the two administrations there was a seven-day washing out period.

The results of the trial were averaged and the mean plasma concentrations after administration to the fed and fasting groups are depicted in FIGS. 1 and 2.

Discussion

The details of the manufacturing process of large dose tablets are a particularly important aspect of the present invention. Large dose tablets present a unique set of problems as the dry mixture of active and inactive ingredients is often not easily compressible.

The present invention discloses a detailed manufacturing procedure which is designed to overcome the difficulties presented in manufacturing tablet or caplet dosages with large doses of the active ingredient. The satisfactory manufacture of large dose tablets or caplets is accomplished by including specific amounts of hydroxypropyl cellulose and other excipients in the tablet or caplet.

Although the plasma concentration results in Example 12 are all based on administration of a single, 1500 mg dose of N-(2-propylpentanoyl) glycinamide, a linear pharmacokinetic response is expected in patients upon administration of other doses. Such a response is expected based on the work of Blotnick et al. with related compounds in phase I studies in which the pharmacokinetics were shown to be dose-independent (Blotnick et al., “The Disposition of Valproyl Glycinamide and Valproyl Glycine in Rats” (1997) Pharmaceutical Research 14(7): 873-878).

What is claimed is:

1. An immediate release solid dosage form comprising the following components:
a) a uniform admixture of:
(i) an active ingredient selected from the group consisting of valproic sodium acid, a pharmaceutically acceptable salt or ester of valproic acid, divalproex sodium, valpromide and a compound having the structure: 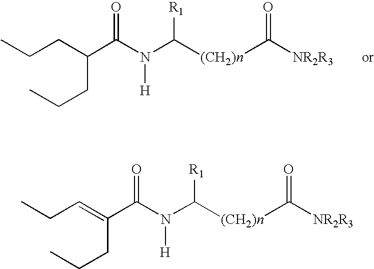
wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and
(ii) a hydroxypropyl cellulose, and
b) a disintegrant.

2. The solid dosage form of claim 1, wherein the solid dosage form is a tablet.

3. The solid dosage form of claim 1 or 2, wherein the uniform admixture of component a) further comprises a filler.

4. The solid dosage form of claim 1 or 2, wherein the solid dosage form further comprises a filler and a lubricant as additional components.

5. The solid dosage form of claim 3, wherein the filler of component a) comprises a microcrystalline cellulose, lactose, a starch, or a combination of two or more of the foregoing.

6. The solid dosage form of claim 5, wherein the filler of component a) comprises a microcrystalline cellulose.

7. The solid dosage form of claim 4, wherein the additional filler comprises a microcrystalline cellulose, lactose, a starch, or a combination of two or more of the foregoing.

8. The solid dosage form of claim 7, wherein the filler comprises a microcrystalline cellulose.

9. The solid dosage form of claim 7, wherein the filler comprises lactose.

10. The solid dosage form of claim 4, wherein the lubricant comprises magnesium stearate, sodium stearyl fumarate, hydrogenated castor oil, hydrogenated soybean oil, polyethylene glycol or a combination of two or more of the foregoing.

11. The solid dosage form of claim 10, wherein the lubricant comprises magnesium stearate.

12. The solid dosage form of claim 10, wherein the lubricant comprises sodium stearyl fumarate.

13. The solid dosage form of claim 1 or 2, wherein the disintegrant of component b) is croscarmellose sodium, sodium starch glycolate or a combination thereof.

14. The solid dosage form of claim 13, wherein the disintegrant of component b) is croscarmellose sodium.

15. The solid dosage form of claim 1 or 2, wherein the active ingredient of component a) is selected from the group consisting of valproic sodium acid, a pharmaceutically acceptable salt or ester of valproic acid, divalproex sodium, valpromide,
N-(2-Propylpentanoyl)glycinamide,
N-(2-propylpentanoyl)glycine-N′-methylamide,
N-(2-propylpentanoyl) glycine-N′-butylamide,
N-(2-propylpentanoyl)leucinamide,
N-(2-propylpentanoyl)alanine-N′-benzylamide,
N-(2-propylpentanoyl)alapinamide,
N-(2-propylpentanoyl)-2-phenylglycinamide,
N-(2-propylpentanoyl)threoninamide,
N-(2-propylpentanoyl)glycine-N′,N′-dimethylamide,
N-(2-propylpent-2-enoyl)glycinamide,
N-(2-propylpent-2-enoyl)alaninamide, and
N-(2-propylpent-2-enoyl)glycine-N′-methylamide.

16. An immediate release tablet comprising the following components:
a) a uniform admixture of:
(i) N-(2-Propylpentanoyl)glycinamide; and
(ii) a hydroxypropyl cellulose; and
b) a disintegrant.

17. The tablet of claim 16, wherein the uniform admixture of component a) further comprises a filler, and the tablet further comprises a filler and a lubricant as additional components.

18. The tablet of claim 17, wherein the filler of component a) comprises a microcrystalline cellulose, lactose, a starch, or a combination of two or more of the foregoing.

19. The tablet of claim 18, wherein the filler of component a) comprises a microcrystalline cellulose.

20. The tablet of claim 18, wherein the additional filler comprises a microcrystalline cellulose, lactose, a starch, or a combination of two or more of the foregoing.

21. The tablet of claim 20, wherein the additional filler comprises a microcrystalline cellulose.

22. The tablet of claim 20, wherein the additional filler comprises lactose.

23. The tablet of claim 17, wherein the lubricant comprises magnesium stearate, sodium stearyl fumarate, hydrogenated castor oil, hydrogenated soybean oil, polyethylene glycol or a combination of two or more of the foregoing.

24. The tablet of claim 23, wherein the lubricant comprises magnesium stearate.

25. The tablet of claim 23, wherein the lubricant comprises sodium stearyl fumarate.

26. The tablet of claim 16, wherein the disintegrant of component b) is croscarmellose sodium, sodium starch glycolate or a combination thereof.

27. The tablet of claim 26, wherein the disintegrant of component b) is croscarmellose sodium.

28. The tablet of claim 16 comprising the following components:
a) a uniform admixture of
from 50 mg/tablet to 1000 mg/tablet N-(2-Propylpentanoyl)glycinamide; and
from 5 mg/tablet to 150 mg/tablet hydroxypropyl cellulose; and
b) from 1 mg/tablet to 100 mg/tablet croscarmellose sodium.

29. The tablet of claim 28, wherein component a) further comprises from 1 mg/tablet to 300 mg/tablet microcrystalline cellulose as an additional component.

30. The tablet of claim 29, wherein the tablet further comprises
from 5 mg/tablet to 500 mg/tablet filler; and
from 0.1 mg/tablet to 20 mg/tablet lubricant.

31. The tablet of claim 16 comprising the following components:
a) a uniform admixture of
from 250 mg/tablet to 500 mg/tablet N-(2-Propylpentanoyl)glycinamide; and
from 25 mg/tablet to 50 mg/tablet hydroxypropyl cellulose; and
b) from 40 mg/tablet to 60 mg/tablet croscarmellose sodium.

32. The tablet of claim 31, wherein component a) further comprises from about 50 mg/tablet to about 100 mg/tablet microcrystalline cellulose as an additional component.

33. The tablet of claim 32, wherein the tablet further comprises
from 100 mg/tablet to 500 mg/tablet filler; and
from 2 mg/tablet to 20 mg/tablet lubricant.

34. The tablet of claim 30 or 33, wherein
the additional filler comprises lactose, microcrystalline cellulose, mannitol or a combination of two or more of the foregoing; and
the lubricant of component b) is magnesium stearate or sodium stearyl fumarate or a combination thereof.

35. The tablet of claim 34 comprising the following components:
a) a uniform admixture of
500 mg/tablet N-(2-Propylpentanoyl) glycinamide;
50 mg/tablet hydroxypropyl cellulose; and
100 mg/tablet a microcrystalline cellulose, and
b) 55 mg/tablet croscarmellose sodium;
145 mg/tablet lactose; and
6 mg/tablet magnesium stearate.

36. The tablet of claim 34 comprising the following components:
a) a uniform admixture of
500 mg/tablet N-(2-Propylpentanoyl) glycinamide;
50 mg/tablet hydroxypropyl cellulose; and
100 mg/tablet a microcrystalline cellulose, and
b) 50 mg/tablet croscarmellose sodium;
145 mg/tablet lactose; and
6 mg/tablet magnesium stearate.

37. The tablet of claim 34, comprising
a) a uniform admixture of:
250 mg/tablet N-(2-Propylpentanoyl) glycinamide;
25 mg/tablet hydroxypropyl cellulose; and
50 mg/tablet microcrystalline cellulose;
b) 450 mg/tablet microcrystalline cellulose;
50 mg/tablet croscarmellose sodium; and
6 mg/tablet magnesium stearate.

38. A method of treating neuropathic pain in a subject in need of such treatment comprising administering to the subject a therapeutically effective dose of the solid dosage form of any one of claims 1-15 or the tablet of any one of claims 16-37 in order to thereby treat the neuropathic pain in the subject.

39. A method of treating a headache disorder in a subject in need of such treatment comprising administering to the subject a therapeutically effective dose of the solid dosage form of any one of claims 1-15 or the tablet of any one of claims 16-37 in order to thereby treat the headache disorder in the subject.

40. A method of treating epilepsy in a subject in need of such treatment comprising administering to the subject a therapeutically effective dose of the solid dosage form of any one of claims 1-15 or the tablet of any one of claims 16-37 in order to thereby treat epilepsy in the subject.

41. A method of controlling seizures in a subject suffering from epilepsy comprising administering to the subject a therapeutically effective dose of the solid dosage form of any one of claims 1-15 or the tablet of any one of claims 16-37 in order to thereby control the seizures in the subject.

42. A method of treating pain in a subject in need of such treatment comprising administering to the subject a therapeutically effective dose of the solid dosage form of any one of claims 1-15 or the tablet of any one of claims 16-37 in order to thereby treat pain in the subject.

43. A method of pain prophylaxis in a subject in need of such treatment comprising administering to the subject a prophylactic dose of the solid dosage form of any one of claims 1-15 or the tablet of any one of claims 16-37 in order to thereby effect pain prophylaxis in the subject.

44. A method of treating mania in bipolar disorder in a subject in need of such treatment comprising administering to the subject a therapeutically effective dose of the solid dosage form of any one of claims 1-15 or the tablet of any one of claims 16-37 in order to thereby treat mania in bipolar disorder in the subject.

45. A method of attenuating bipolar mood swings in a subject suffering from bipolar disorder comprising administering to the subject a therapeutically effective dose of the solid dosage form of any one of claims 1-15 or the tablet of any one of claims 16-37 in order to thereby attenuate the bipolar mood swings in the subject.

46. A process for preparing the solid dosage form of claim 1 or 2, comprising the steps of:
a) admixing predetermined amounts of
(i) an active ingredient selected from the group consisting of valproic sodium acid, a pharmaceutically acceptable salt or ester of valproic acid, divalproex sodium, valpromide and a compound having the structure: 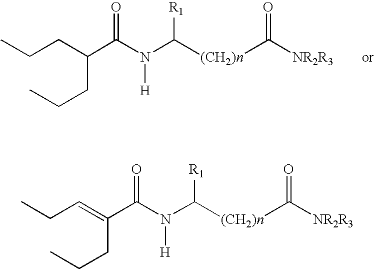
wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and
(ii) a hydroxypropyl cellulose;
b) admixing the uniform mixture of step a) with a predetermined amount of a disintegrant; and
c) compressing the mixture of step b) to form the tablet.

47. The process of claim 46, wherein step b) further comprises admixing the uniform mixture with predetermined amounts of a filler and a lubricant.

48. The process of claim 47, wherein the filler of step b) is microcrystalline cellulose, anhydrous dicalcium phosphate, lactose or a combination of two or more of the foregoing.

49. The process of claim 48, wherein the filler is lactose.

50. The process of claim 48, wherein the filler is a microcrystalline cellulose.

51. The process of claim 47, wherein the lubricant is magnesium stearate or sodium stearyl fumarate or a combination thereof.

52. The process of claim 51, wherein the lubricant is magnesium stearate.

53. The process of claim 51, wherein the lubricant is sodium stearyl fumarate.

54. The process of claim 47, wherein the disintegrant of step b) is croscarmellose sodium, sodium starch glycolate or a combination thereof.

55. The process of claim 54, wherein the disintegrant of step b) is croscarmellose sodium.

56. Use of an active ingredient selected from the group consisting of valproic sodium acid, a pharmaceutically acceptable salt or ester of valproic acid, divalproex sodium, valpromide and a compound having the structure: 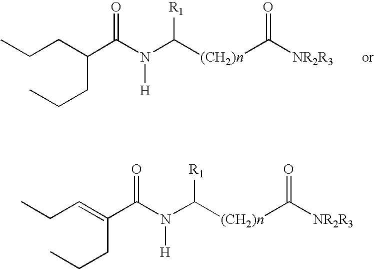
wherein R_{1, R}₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-16 or the tablet of any one of claims 16-37 for use in treating a headache disorder in a subject.

57. Use of an active ingredient selected from the group consisting of valproic sodium acid, a pharmaceutically acceptable salt or ester of valproic acid, divalproex sodium, valpromide and a compound having the structure: 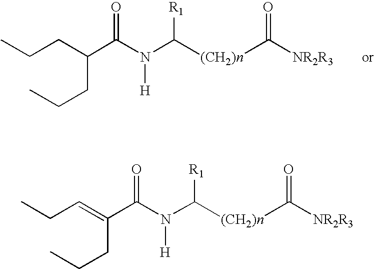
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-16 or the tablet of any one of claims 16-37 for use in treating neuropathic pain in a subject.

58. Use of an active ingredient selected from the group consisting of valproic sodium acid, a pharmaceutically acceptable salt or ester of valproic acid, divalproex sodium, valpromide and a compound having the structure: 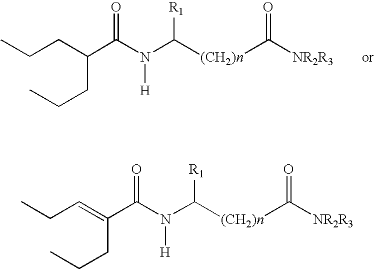
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-16 or the tablet of any one of claims 16-37 for use in treating epilepsy in a subject.

59. Use of an active ingredient selected from the group consisting of valproic sodium acid, a pharmaceutically acceptable salt or ester of valproic acid, divalproex sodium, valpromide and a compound having the structure: 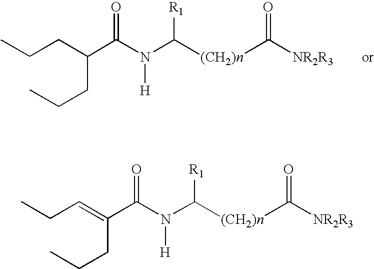
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-16 or the tablet of any one of claims 16-37 for use in controlling seizures in a subject suffering from epilepsy.

60. Use of an active ingredient selected from the group consisting of valproic sodium acid, a pharmaceutically acceptable salt or ester of valproic acid, divalproex sodium, valpromide and a compound having the structure: 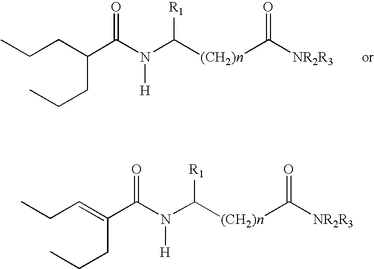
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release tablet of any one of claims 1-32 for use in treating mania in bipolar disorder in a subject.

61. Use of an active ingredient selected from the group consisting of valproic sodium acid, a pharmaceutically acceptable salt or ester of valproic acid, divalproex sodium, valpromide and a compound having the structure: 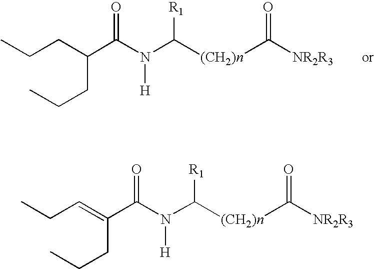
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-16 or the tablet of any one of claims 16-37 for use in attenuating bipolar mood swings in a subject suffering from bipolar mood disorder.

62. Use of an active ingredient selected from the group consisting of valproic sodium acid, a pharmaceutically acceptable salt or ester of valproic acid, divalproex sodium, valpromide and a compound having the structure: 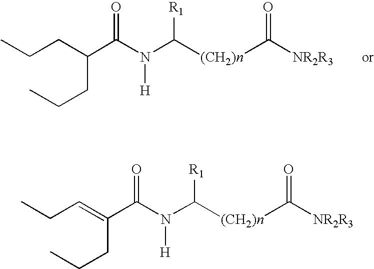
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-15 or the tablet of any one of claims 16-37 for use in treating pain in a subject.

63. Use of an active ingredient selected from the group consisting of valproic sodium acid, a pharmaceutically acceptable salt or ester of valproic acid, divalproex sodium, valpromide and a compound having the structure: 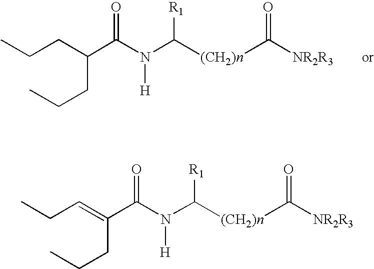
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing the immediate release solid dosage form of any one of claims 1-15 or the tablet of any one of claims 16-37 for use in effecting pain prophylaxis in a subject.

64. The immediate release solid dosage form of any one of claims 1-15 or tablet of any one of claims 16-37 for use in treating a headache disorder in a subject.

65. The immediate release solid dosage form of any one of claims 1-15 or tablet of any one of claims 16-37 for use in treating neuropathic pain in a subject.

66. The immediate release solid dosage form of any one of claims 1-15 or tablet of any one of claims 16-37 for use in treating epilepsy in a subject.

67. The immediate release solid dosage form of any one of claims 1-15 or tablet of any one of claims 16-37 for use in controlling seizures in a subject suffering from epilepsy.

68. The immediate release solid dosage form of any one of claims 1-15 or tablet of any one of claims 16-37 for use in treating mania in bipolar disorder in a subject.

69. The immediate release solid dosage form of any one of claims 1-15 or tablet of any one of claims 16-37 for use in attenuating bipolar mood swings in a subject suffering from bipolar disorder.

70. The immediate release solid dosage form of any one of claims 1-15 or tablet of any one of claims 16-37 for use in treating pain in a subject.

71. The immediate release solid dosage form of any one of claims 1-15 or tablet of any one of claims 16-37 for use in effecting pain prophylaxis in a subject.
